(19) United States
(12) Patent Application Publication
(10) Pub. No.: US 2010/0185015 A1
(43) Pub. Date: Jul. 22, 2010

(54) Process for preparing biaryls

(75) Inventors: Alexander Straub, Wuppertal (DE); Norbert Lui, Odenthal (DE); Juergen Wieschemeyer, Bergisch Gladbach (DE); Ulrich Kloettschen, Dormagen (DE); Eric Wihelus Petrus Damen, Wijchen (NL)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim (DE)

(21) Appl. No.: 12/666,013

(22) PCT Filed: Jun. 27, 2008
(86) PCT No.: PCT/EP2008/005270

§ 371 Date: Dec. 22, 2009

(30) Foreign Application Priority Data
Jun. 29, 2007 (EP) 07111438.3

(51) Int. Cl. C07C 209/68

(52) U.S. Cl. 564/442

(57) ABSTRACT

The present invention relates to a process for preparing biaryls using catalysts based on palladium compounds with phosphine ligands.

The present invention relates to a process for preparing biaryls using catalysts based on palladium compounds with phosphine ligands.

Biaryl compounds, in particular biphenyl compounds, are industrially important as fine chemicals, intermediates for pharmaceuticals, optical brighteners and agrochemicals.

A method which is frequently employed for the synthesis of biaryls on a laboratory scale is the Suzuki reaction in which iodoaromatics or bromoaromatics or in exceptional cases chloroaromatics are reacted with arylboronic, vinylboronic or alkylboronic acid derivatives in the presence of palladium catalysts. Review articles describing this methodology may be found for example in N. Miyaura, A. Suzuki, Chem. Rev. 1995, 95, 2457 and Bellina, F. et al. Synthesis 2004, 2419. A review of the use of trialkylphosphine ligands in the Pd-catalyzed reaction of chloroaromatics may be found in Littke, A. F. & Fu, G. C. Angew. Chem. 2002, 114, 4350.

Catalysts used for the purposes of the Suzuki reaction are in general palladium and nickel compounds. Despite the economic advantage of nickel catalysts (cf. A. F. Indolese, Tetrahedron Lett. 1997, 38, 3513), palladium catalysts are preferred over nickel catalysts because of their lower toxicity and their greater tolerance toward functional groups. When using palladium catalysts, both palladium(II) and palladium(0) complexes are employed in Suzuki reactions (cf. M. Beller, H. Fischer, W. A. Herrmann, K. Öfele, C. Broβmer, Angew. Chem. 1995, 107, 1992). According to the literature, coordinatively unsaturated 14- and 16-electron palladium(0) species which are stabilized with donor ligands such as phosphanes are formulated as catalytically active species. In particular when using relatively low-cost starting materials such as aryl bromides or aryl chlorides it is necessary to add stabilizing ligands in order to achieve a satisfactory catalytic activation of the starting materials. A significant disadvantage of the Suziki reactions described is that satisfactory catalytic turnover numbers (=TON) can only be achieved with expensive starting materials such as iodoaromatics and activated (i.e. electron-deficient) bromoaromatics. Otherwise, when using deactivated (i.e. electron-rich) bromoaromatics or chloroaromatics, large amounts of catalysts, usually from 1 to 5 mol %, have to be added in order to achieve industrially acceptable conversions.

Furthermore, ortho-substituted haloaromatics have a lower reactivity owing to the greater steric hindrance. Haloanilines can also be problematic reactants because they can additionally act as ligands for the catalyst.

The reaction of fluorohaloanilines with substituted boronic acids in the presence of a catalyst is described in WO 03/070705.

In this context, WO 00/61531 describes the use of catalysts with phosphite-containing ligands.

EP 1 186 583 teaches the use of supported Pd catalysts.

EP 1 064 243 and WO 0116057 teach the use of allylic Pd complexes, in EP 0 690 046 palladacycles are used as catalyst.

All the processes mentioned involve the use of palladium complexes which are expensive or can be prepared only in a complex manner or require the use of an excess of arylboronic acid in order to achieve a good yield. This increases the cost of the process not only because of the loss of valuable arylboronic acid, but also because of more complicated purification and isolation processes which are necessary to separate excess boronic acid and by-products resulting therefrom such as deboronated aromatics and homocoupling products.

WO 2006/092429 describes the reaction of aromatic borinic acids with aryl halides in aqueous solvent systems inter alia in the presence of trialkylphosphines. However, the synthesis of borinic acids is not always easy.

WO 2006/024388 describes an alternative process for preparing biphenylamines by reacting substituted phenylacetamides with butynols and subsequent Diels-Alder reaction with thiophene dioxides.

WO 2005/123689 describes the preparation of 3,4-(dichlorophenyl)aniline by Suzuki coupling using tetrakis(triphenylphosphine)palladium(0).

The reactivity of the boronic acid or borinic acid used also has a decisive influence on the course of the Suzuki reaction; in particular aromatics deactivated by electron-withdrawing substituents may react more slowly and form homocoupling products. However, this problem is hardly taken into consideration in the methodologically-oriented literature because a large excess of boronic acid is commonly used here, and the yields are only based on the conversion of the haloaromatic. A further disadvantage of the processes previously described in the prior art is therefore the competing homocoupling reaction of the haloaromatics which produces toxic polyhalogenated biphenyls.

Moreover, simple catalyst recycling is not possible owing to the complexity of the reaction mixtures, so that catalyst costs also generally stand in the way of industrial implementation. Catalyst systems based on water-soluble phosphanes do give satisfactory catalyst activities in the industrially important reaction of 2-chlorobenzonitrile with p-tolylboronic acid, but the catalysts comprise expensive sulphonated phosphanes.

It is an object of the present invention to provide a novel process for preparing biaryls which does not exhibit the disadvantages of the known processes, is suitable for industrial implementation and gives biaryls in high yield, high purity and optimum catalyst productivity.

This object is achieved by a process for preparing monofunctional, bifunctional and/or polyfunctional biaryls of the general formula (I)

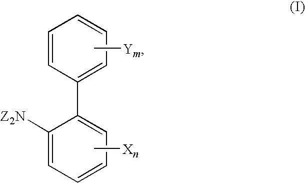

in which
Z is hydrogen or oxygen
n is an integer selected from 1, 2 or 3 and
X is independently selected from the group consisting of F, Cl, C₁-C₄-alkyl and C₁-C₄-alkyloxy groups;
m is an integer selected from 0, 1, 2, 3, 4 or 5 and
Y is independently selected from halogen, C₁-₄-alkyl, C₁-C₄-alkyloxy, C₁-₄-haloalkyl, C₁-₄-haloalkoxy and hydroxy groups,
   by reacting haloaromatics of the general formula (II)

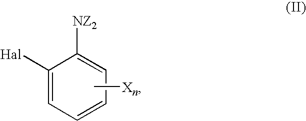

in which
Hal is a halogen atom
with
(a) at least one boronic acid of the general formula (III-a)

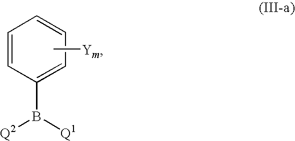

in which
Q¹ and Q² are hydroxyl groups (—OH)
or with the anhydrides, dimers and trimers formed from the boronic acids of the formula (III-a);
or with at least one boronic acid derivative of the formula (III-a),
in which
Q¹ and Q² are independently selected from the group consisting of F, Cl, Br, I, C₁-₄-alkyl, C₆-₁₀-aryl, C₁-C₄-alkyloxy and C₆-₁₀-aryloxy groups;
   or with
   (b) at least one cyclic boronic ester of the formula (III-b)

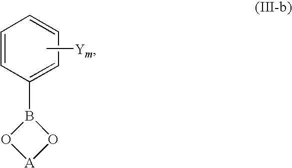

in which
A is selected from radicals selected from the group consisting of —CH₂—CH₂, —C(CH₃)₂—C(CH₃)₂—, —CH₂—C(CH₃)₂—CH₂—;
   or with
   (c) at least one boronate of the general formula (III-c)

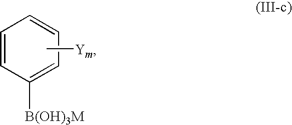

in which
M⁺ is a cation; or
(d) at least one borinic acid of the general formula (III-d)

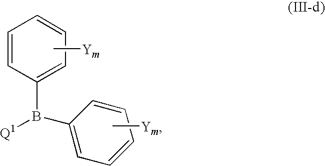

in which Y, Q¹ and m are as defined above,
in the presence of at least one palladium phosphine complex, wherein the phosphine group is substituted by at least one branched C₃₋₈-alkyl group.

In the context of the present invention, the term halogens (X) comprises, unless otherwise defined, those elements which are selected from the group consisting of fluorine, chlorine, bromine and iodine, fluorine, chlorine and bromine being preferably used and fluorine and chlorine being particularly preferably used.

Optionally substituted groups can be monosubstituted or polysubstituted, it being possible for the substituents in polysubstitutions to be identical or different.

Alkyl groups substituted with one or more halogen atoms (—X) are selected, for example, from trifluoromethyl (CF₃), difluoromethyl (CHF₂), CF₃CH₂, CICH₂, CF₃CCl₂.

In the context of the present invention, alkyl groups are, unless otherwise defined, linear, branched or cyclic hydrocarbon groups which can optionally contain one, two or more heteroatoms selected from O, N, P and S. In addition, the alkyl groups according to the invention can optionally be substituted by additional groups selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl-group, particularly preferably a C₃₋₈-alkyl group which can contain one or more heteroatoms selected from N, O, P and S.

The definition of C₁-C₁₂-alkyl comprises the largest range defined herein for an alkyl group. Specifically, this definition comprises for example the meanings methyl, ethyl, n-propyl, isopropyl, n-butyl, isobutyl, sec-butyl and t-butyl, n-pentyl, n-hexyl, 1,3-dimethylbutyl, 3,3-dimethylbutyl, n-heptyl, n-nonyl, n-decyl, n-undecyl, n-dodecyl, cyclobutyl, cyclohexyl, cycloheptyl and cyclooctyl.

In the context of the present invention, aryl groups are, unless otherwise defined, aromatic hydrocarbon groups which may contain one, two or more heteroatoms selected from O, N, P and S and can optionally be substituted by additional groups selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group which may contain one or more heteroatoms selected from N, O, P and S.

The definition of C5-18-aryl comprises the largest range defined herein for an aryl group having 5 to 18 framework atoms, where the C atoms may be replaced by heteroatoms. Specifically, this definition comprises for example the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In the context of the present invention, arylalkyl groups (aralkyl groups) are, unless otherwise defined, alkyl groups substituted by aryl groups which may contain a C₁₋₈-alkylene chain and may be substituted in the aryl framework or in the alkylene chain by one or more heteroatoms selected from O, N, P and S and optionally by additional groups selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (—CONR₂′) groups, where R′ is hydrogen or a C₁₋₁₂-alkyl group, preferably a C₂₋₁₀-alkyl group, particularly preferably a C₃₋₈-alkyl group, which can contain one or more heteroatoms selected from N, O, P and S.

The definition of C₇₋₁₉-aralkyl group comprises the largest range defined herein for an aryl alkylgroup having a total of 7 to 19 atoms in the framework and alkylene chain. Specifically, this definition comprises for example the meanings benzyl and phenyl ethyl.

In the context of the present invention, alkylaryl groups (alkaryl groups) are, unless otherwise defined, aryl groups substituted by alkyl groups which may contain a C₁₋₈-alkylene chain and may be substituted in the aryl framework or the alkylene chain by one or more heteroatoms selected from O, N, P and S and optionally by additional groups selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide (CONR₂′) groups, where R′ is hydrogen or a C₁₋₁₂-alkyl group, preferably a C₂₋₁₀-alkyl group, particularly preferably a C₃₋₈-alkyl group, which may contain one or more heteroatoms selected from N, O, P and S.

The definition of C₇₋₁₉-alkylaryl group comprises the largest range defined herein for an alkylaryl group having in total 7 to 19 carbon atoms in the framework and alkylene chain. Specifically, this definition comprises for example the meanings tolyl, 2,3-, 2,4-, 2,5-, 2,6-, 3,4- or 3,5-dimethylphenyl.

The alkyl, alkenyl, alkynyl, aryl, alkaryl and aralkyl groups can furthermore contain one or more heteroatoms which, unless otherwise defined, are selected from N, O, P and S. The heteroatoms replace the carbon atoms indicated. The compounds according to the invention may exist, if appropriate, as mixtures of different possible isomeric forms, in particular of stereoisomers, such as E- and Z-isomers, threo and erythro isomers and optical isomers, but also tautomers, if appropriate. Both the E and Z isomers and also the threo and erythro isomers and also the optical isomers, any mixtures of these isomers and the possible tautomeric forms are disclosed and claimed.

In the context of the present invention, the haloaromatics of the formula (II) are fluoroaromatics, chloroaromatics, bromoaromatics or iodoaromatics. In a preferred embodiment, the haloaromatics of the formula (II) are selected from anilines (Z=H); particular preference is given to 2-bromo-4-fluoroaniline.

In the boronic acids of the formula (III-a) or their derivatives, Q′ and Q² together with the boron atom and one or two oxygen atoms may form a five- or six-membered ring which can be substituted with additional methyl groups.

Particular preference is given to boron compounds of the formula (III-a) with Q₁, Q₂₌OH and also boronic acids.

Alternatively, the anhydrides, dimers and trimers formed from boronic acids of the formula (III-a) or derivatives thereof may be used as coupling partners.

The boronic acids of the formula (III-a) or derivatives thereof can be obtained by reacting arylmagnesium halides (Grignard reagents) with trialkyl borates, preferably in a solvent such as THF.

To suppress the competing formation of arylborinic acids, the reaction must be carried out at low temperatures (−60° C.), and an excess of reagents is to be avoided, as described in R. M. Washburn et al., Organic Syntheses Collective Vol. 4, 68 or in Boronic Acids, edited by Dennis G. Hall, Wiley-VCH 2005, p. 28ff.

Preference is furthermore given to cyclic boronic esters of the formula (III-b).

A very particularly preferred embodiment of the present invention relates to boronic acids of the general formula (III-a) with m=2; Y=3-Cl and 4-Cl, Q₁, Q₂=OH and their dimers, trimers and anhydrides.

The cyclic boronic esters of the general formula (III-b) are preferably those with Y=Cl and m=2, particularly preferably Y=3-Cl and 4-Cl.

The cyclic boronic esters of the general formula (III-b) can be prepared as described in Boronic Acids, edited by Dennis G Hall, Wiley-VCH 2005, p. 28ff.

In the context of the present invention, the boronates of the general formula (III-c) contain a cation (M⁺) which is selected from alkali metals and alkaline earth metals, such as Li, Na, K, Cs, Mg, Ca and Ba, or from tetraalkylammonium cations, such as NMe₄⁺, NEt₄⁺, NBut₄⁺, or from trialkylammonium cations such as HNEt₃⁺. Boronates of the general formula (III-d) which are preferably used are those with Y=Cl, m=2, M⁺=Na, K, Mg; particular preference is given to those with Y=3-Cl and 4-Cl.

Boronates of the formula (III-c) can be obtained as described in Serwatowski et al., Tetrahedron Lett. 44, 7329 (2003).

Borinic acids of the formula (III-d) can be obtained as described in WO 2007/138089.

The boron compounds are preferably reacted in the presence of at least one solvent which is selected for example from the group consisting of water, aliphatic ethers, optionally halogenated aromatic or aliphatic hydrocarbons, alcohols, esters, aromatic or aliphatic nitriles and dipolar aprotic solvents such as dialkylsulfoxides, N,N-dialkylamides of aliphatic carboxylic acids or alkylated lactams.

Particular preference is given to solvents selected from the group consisting of THF, dioxane, diethyl ether, diglyme, methyl tert-butyl ether (MTBE), tert-amyl methyl ether (TAME), dimethyl ether (DME), 2-methyl-THF, acetonitrile, butyronitrile, toluene, xylenes, mesitylene, anisole, ethyl acetate, isopropyl acetate, methanol, ethanol, propanol, butanol, ethylene glycol, ethylene carbonate, propylene carbonate, N,N-dimethylacetamide, N,N-dimethylformamide, N-methylpyrrolidone, water and mixtures thereof.

Very particular preference is given to mixtures comprising the environmentally friendly solvent water.

It has also been observed that the addition of small amounts of water to the organic solvents contributes to a substantial suppression of the competing homocoupling reaction.

Owing to the solubilities of the starting materials and the resulting products, however, it is generally not possible to completely dispense with the organic (apolar) solvent. Therefore the organic solvents are preferably used as cosolvents.

The solvent mixtures of the invention may contain between 0.1 and 95% by volume and preferably between 1 and 60% by volume of water, based on the mixture of water and the organic solvent.

Since an acid is formed in the reaction, it is advantageous to neutralize the resulting acid by addition of a base. The base may either be present from the start or may be added continuously during the reaction (semi-batch process).

Bases which are suitable according to the present invention are for example primary, secondary and tertiary amines such as alkylamines, dialkylamines, trialkylamines, each of which may be alicyclic or open-chain; alkali metal and alkaline earth metal salts of aliphatic and/or aromatic carboxylic acids, such as acetates, propionates or benzoates; alkali metal and alkaline earth metal carbonates, hydrogencarbonates, phosphates, hydrogenphosphates and/or hydroxides; and metal alkoxides, in particular alkali metal or alkaline earth metal alkoxides, such as sodium methoxide, potassium methoxide, sodium ethoxide, magnesium methoxide, calcium ethoxide, sodium tert-butoxide, potassium tert-butoxide or alkali metal isoamylates. The base is preferably a carbonate, hydroxide or phosphate of lithium, sodium, potassium, calcium, magnesium or caesium. Particular preference is given to NaOH, KOH, potash and soda.

Apart from the neutralization of the resulting acid, the base used may also have a positive influence on the course of the reaction by activating the arylboronic acid to form anionic boronate species. Apart from the abovementioned bases, such an activation can also be achieved by addition of fluoride salts such as CaF, NaF, KF, LiF, CsF or (C,-C,)-alkyl, NF.

The palladium catalysts used are generally produced in situ from at least one palladium(II) salt or a palladium(0) compound and the corresponding phosphine ligands. However, they may also be used directly as palladium(0) compound without reducing the initial catalytic activity.

Suitable palladium sources are for example selected from the group consisting of palladium trifluoroacetate, palladium fluoroacetylacetonate, Pd(OAc)₂, Pd(OCOCH₂CH₃)₂, Pd(OH)₂, PdCl₂, PdBr₂, Pd(acac)₂ (acac=acetylacetonate), Pd(NO₃)₂, Pd(dba)₂, Pd₂ dba₃ (dba=dibenzylideneacetone), Pd(CH₃CN)₂Cl₂, Pd(PhCN)₂Cl₂, Li[PdCl₄], Pd/C or palladium nanoparticles.

A preferred embodiment envisages the use of methyldi(C₃₋₈-alkyl)phosphine or tri(C₃₋₈-alkyl)phosphine ligands which are branched in the alkyl part or salts thereof, particularly preferably of methyldi(tert-butyl)phosphine and tri(tert-butyl)phosphine, as ligand.

The trialkylphosphine may also be used as trialkylphosphonium salt such as tetrafluoroborate (Org. Lett. 2001, 3, 4295), perchlorate or hydrogen sulphate and released therefrom in situ with a base.

The molar ratio of palladium to the phosphine ligand should be between 4:1 and 1:100 and is preferably between 1:1 and 1:5, particularly preferably between 1:1 and 1:2.

According to the invention, it is also possible to use Pd[P(t-But)₃]₂ directly, the preparation of which is described in (J. Amer. Chem. Soc. 1976, 98, 5850; J. Amer. Chem. Soc. 1977, 99, 2134; J. Am. Chem. Soc. 2001, 123, 2719).

A further preferred embodiment involves the use of 1,1-bis(di-t-butylphosphino)ferrocene (D.t.BPF) as ligand on the palladium.

When carrying out the reaction, the catalyst system (Pd+ligand) can be added together or separately either at room temperature or at an elevated temperature. The system can be prepared separately, immediately before the reaction is carried out, by combining a Pd salt and the ligand, or it can be purchased in crystalline form. Also possible is the direct addition of the ligand and then of the palladium salt to the batch (in situ process).

According to the present invention, the haloaromatics of the formula (II) and the boron compounds of the formulae (III-a) to (III-c) are used in an equimolar ratio. Alternatively, it is also possible to use one of the two components (II or III), preferably the boron compounds (III-a) to (III-c), in excess. It is also possible to carry out the reaction under metering control, in which case one of the two reaction components is slowly metered in during the reaction. For this purpose, preference is given to using e.g. a solution of the boronic acid or the boronate, while the halogen component, the catalyst and the base, if used, are initially charged.

The reaction is generally carried out at a temperature between 10 and 200° C., preferably between 20 and 140° C., and at a pressure of up to 100 bar, preferably at a pressure between atmospheric pressure and 40 bar.

The reaction is preferably carried out in the absence of atmospheric oxygen under a protective gas atmosphere, such as under argon or nitrogen atmosphere.

Owing to the catalyst activities and stabilities, the process of the invention makes it possible to use extremely small amounts of catalyst so that the catalyst costs are not limiting, in contrast to the known Suzuki reactions for the corresponding process.

In the process of the invention, catalyst contents of from 0.0001 to 5 mol %, particularly preferably <0.1 mol %, based on the halo component, are used.

In most cases, the catalyst may remain in the final product since the catalyst amounts are small. Alternatively, the resulting biaryls can be purified by filtration, e.g. over Celite.

The following examples illustrate the process of the invention without limiting it thereto.

### EXAMPLES OF THE PREPARATION OF 3′,4′-DICHLORO-5-FLUOROBIPHENYL-2-AMINE

The examples demonstrate that the process according to the invention makes it possible to achieve high yields using a catalyst and ligand amount of less than 0.1 mol % while producing very small amounts (<1% instead of e.g. 10%) of homocoupling products of the boronic acid.

### Using Commercial Catalyst (0.01 mol %) in Acetonitrile-Water.

29.5 g (213.5 mmol) of potash are added to 210 ml of water, 22.3 g (94.2%, 110.1 mmol) of 3,4-dichlorophenylboronic acid (contained 1% of 3,4-dichlorobromobenzene and 0.3% of PCB077) and 150 ml acetonitrile are added, the mixture is stirred for 25 minutes, and 19.16 g (99.2%, 100 mmol) of 2-bromo-4-fluoroaniline in 50 ml acetonitrile are added. The solution is evacuated and charged with argon six times, and then 6 mg of bis(tri-t-butylphosphine)palladium are added. The mixture is stirred under argon at 67-69° C. for 20 hours and left to cool to room temperature, 150 ml of ethyl acetate are added, the organic phase is separated, the mixture is extracted twice with 50 ml of ethyl acetate each time, and the combined organic phases are evaporated to give 29.25 g of an oil which crystallizes. Purity (HPLC) 86.2%; yield 99.1%.

### Using Freshly Prepared Catalyst (0.01 Mol %)

548 mg of a 12.9% solution of tri-t-butylphosphine in toluene are dissolved in 20 ml of THF. 4 ml of a solution of 69 mg of Pd2 dba3 in 15 ml of THF are added to 2.28 ml of this solution, and the mixture is stirred for 10 minutes. 1.57 ml of the above catalyst solution are added to an argon-saturated solution of 19.35 g (99.2%, 100 mmol) of 2-bromo-4-fluoroaniline, 22.5 g (94.2%, 110.1 mmol) of 3,4-dichlorophenylboronic acid and 29.3 g (212 mmol) of potash in 115 ml water and 115 ml toluene, and the mixture is stirred at 67-69° C. for 17 h. The organic phase is separated off and the aqueous phase is washed once with 50 ml of toluene. The combined organic phases are evaporated under reduced pressure, leaving 27.93 g of an oil which crystallizes. Purity (GCMS): 89%. Yield: 89.8%. PCB<0.1%.

Using 1,1-bis(di-t-butylphosphino)ferrocene (D.t.BPF)

### In Toluene/Water

8.15 g (97%, 0.042 mol) of 3,4-dichlorophenylboronic acid are initially charged in 50 g of water and 50 g toluene. Subsequently 18.7 g (0.085 mol) of potassium phosphate and 8.2 g (98%, 0.042 mol) of 2-bromo-4-fluoroaniline are added. After blanketing with nitrogen, 0.014 g (0.00002 mol) of 1,1′-bis(di-tert-butylphosphino)ferrocenepalladium dichloride are added, and the mixture is stirred for two hours at 79-81° C. The organic phase is separated off and evaporated under reduced pressure, leaving 10.5 g of an oil which crystallizes. Purity (HPLC): 97%, yield: 95%. PCB<1%.

### In THF/Water

8.15 g (98%, 0.042 mol) of 2-bromo-4-fluoroaniline and 7.8 g (97%, 0.040 mol) of 3,4-dichlorophenylboronic acid are initially charged in 20 g of water and 50 g of tetrahydrofuran. After blanketing with nitrogen, 0.014 g (0.00002 mol) of 1,1′ bis(di-tert-butylphosphino)ferrocenepalladium chloride are added, and the mixture is heated to 65-67° C. Then a solution of 13.4 g (99.8%, 0.1262 mol) of sodium carbonate in 30 g of water is added dropwise within one hour, and after the addition is complete, the mixture is stirred for an additional two hours at 65-67° C. The organic phase is separated off and evaporated under reduced pressure, leaving 10.5 g of an oil which crystallizes. Purity (HPLC): 96%, yield: 93.7%. PCB<1%.

Semi-batch in toluene/water with NaOH

85.46 g (99.2%, 0.446 mol) of 2-bromo-4-fluoroaniline and 90.2 g (0.473 mol) of 3,4-dichlorophenylboronic acid are initially charged in 200 g of water and 565 g of toluene. After blanketing with nitrogen, the mixture is heated to 85° C., and 25.9 mg (0.02 mol %) of tri-tert-butylphosphine tetrafluoroborate in 5 ml of water and 27.2 mg (0.02 mol %) palladium(II) acetylacetonate in 5 ml of toluene are added.

Then a 10% solution of sodium hydroxide is added dropwise within about two hours such that a pH of 8-8.5 is maintained. This requires about 1.2-1.4 equivalents. After complete conversion is detected using HPLC, the organic phase is separated off and evaporated under reduced pressure, leaving 10.5 g of an oil which crystallizes. Purity (HPLC): 96%, yield: 93.7%. PCB<1%. Further purification is carried out by precipitating with concentrated HCl, washing the precipitated hydrochloride with toluene and releasing using toluene/MeOH/water/NaOH. The organic phase is evaporated under reduced pressure which gives the desired product in the form of an oil (157.9 g; content (HPLC against standard): 71.2%; yield 89.5%).

1. Process for preparing a monofunctional, bifunctional and/or polyfunctional biaryl formula (I) 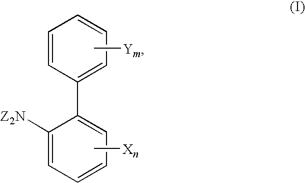
in which
Z is hydrogen or oxygen
n is an integer selected from 1, 2 or 3 and
X is independently selected from the group consisting of F, Cl, C₁-C₄-alkyl and
C₁-C₄-alkyloxy groups;
m is an integer selected from 0, 1, 2, 3, 4 or 5 and
Y is independently selected from halogen, C₁₋₄-alkyl, C₁-₄-alkoxy, C₁₋₄-haloalkyl, C₁₋₄-haloalkoxy and hydroxy groups,
by reacting a haloaromatic of formula (II) 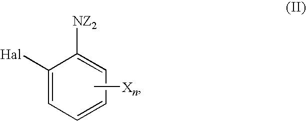
in which
Hal is a halogen atom
with
(a) at least one boronic acid of formula (III-a) 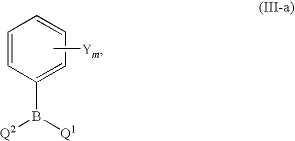
in which
Q¹ and Q² are hydroxyl groups (—OH)
or with an anhydride, dimmer and/or trimer formed from a boronic acid of formula (III-a);
or with at least one boronic acid derivative of the formula (III-a),
in which
Q¹ and Q² are independently selected from the group consisting of F, Cl, Br, I, C₁₋₄-alkyl, C₆₋₁₀-aryl, C₁-₄-alkoxy and C₆₋₁₀-aryloxy groups;
or with
(b) at least one cyclic boronic ester of the formula (III-b) 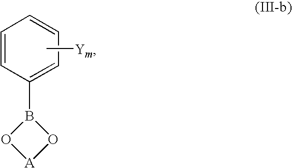
in which
A is a radical selected from the group consisting of —CH₂—CH₂—, —C(CH₃)₂—C(CH₃)₂—, and —CH₂—C(CH₃)₂—CH₂—;
or with
(c) at least one boronate of the general formula (III-c) 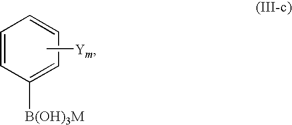
in which
M⁺ is a cation; or
(d) at least one borinic acid of the general formula (III-d) 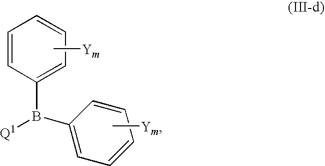
in the presence of at least one palladium phosphine complex, wherein a phosphine group thereof is substituted by at least one branched C₃₋₈-alkyl group.

2. Process according to claim 1, wherein Z=hydrogen, n=1, X=5-F, m=2, Y=3′-Cl and 4′-Cl, Hal=Br and Q¹ and Q² are each hydroxyl groups.

3. Process according to claim 1 wherein the process is carried out in the presence of an organic solvent.

4. Process according to claim 3, wherein the solvent comprises between 0.1 and 95% by volume of water, based on a mixture of water and the organic solvent.

5. Process according to one of claim 1 wherein the haloaromatic of formula (II) is 2-bromo-4-fluoroaniline.

6. Process according to claim 1 the palladium complex is at least one selected from the group consisting of bis(tri-tert-butylphosphine)palladium, bis[methyldi(tert-butyl)phosphine]palladium and [1,1-bis(di-t-butylphosphino)ferrocene]palladium.

7. Process according to claim 1 wherein the palladium complex is generated in situ by adding a palladium source and a Pd ligand.

8. Process according to claim 6, wherein the ligand is tri(tert-butyl)phosphine or a salt thereof.

9. Process according to claim 6, wherein the ligand is methyldi(tert-butyl)phosphine or a salt thereof.

10. Process according to claim 8, wherein a corresponding hydrogen tetrafluoroborate salt (HBF₄ salt) is used.

11. Process according to claim 7 wherein the palladium source is palladium acetylacetonate or palladium dibenzylideneacetonate.

12. Process according to claim 1, wherein the boronic acid of formula (III-a) is 3,4-dichlorophenylboronic acid.

13. Process according to claim 1, wherein in that the borinic acid of the formula (III-d) is bis(3,4-dichlorophenyl)borinic acid.

14. Process according to claim 8 wherein the palladium source is palladium acetylacetonate or palladium dibenzylideneacetonate.

15. Process according to claim 9 wherein the palladium source is palladium acetylacetonate or palladium dibenzylideneacetonate.

16. Process according to claim 10 wherein the palladium source is palladium acetylacetonate or palladium dibenzylideneacetonate.

17. Process according to claim 2, wherein the palladium complex is generated in situ by adding a palladium source and a Pd ligand.

18. Process according to claim 3, wherein the palladium complex is generated in situ by adding a palladium source and a Pd ligand.

19. Process according to claim 4, wherein the palladium complex is generated in situ by adding a palladium source and a Pd ligand.

20. Process according to claim 5, wherein the palladium complex is generated in situ by adding a palladium source and a Pd ligand.
